(19) United States
(12) Patent Application Publication
(10) Pub. No.: US 2018/0265837 A1
(43) Pub. Date: Sep. 20, 2018

(54) System and method for efficiently growing algae using fungus

(71) Applicant: McKenna Kristin Loop, Chandler, AZ (US)

(72) Inventor: McKenna Kristin Loop, Chandler, AZ (US)

(21) Appl. No.: 15/530,893

(22) Filed: Mar. 17, 2017

(51) Int. Cl. C12N 1/38

(52) U.S. Cl. CPC C12N 1/38; C02F 3/322

(57) ABSTRACT

A new method of using fungus to assist the growth of algae.

### BACKGROUND OF THE INVENTION

Algae are rich in hydrocarbons (lipids), which can be used to produce fuel. Algae biofuels have the potential to provide many benefits for the growing world; however, the method of growing that algae must be as efficient as possible so as to make algae biofuels economically competitive with other types of fuels. Current methods are inefficient and expensive.

The purpose of this invention is to establish a new method for growing algae before extracting the hydrocarbons from the algae. One of the current best methods growing algae uses a BG11 medium. This method yields a final cell concentration was 1.16×10⁴ cells/ml. The new method proposed here, which adds the use of fungus to assist the growth process, yields a final cell concentration of 5.28×10⁴ cells/ml. This is roughly a 500% improvement in cell concentration, and this method of growth requires no expensive gas exchange equipment. This is projected to lower the cost of algae biofuels to under $3/gallon, thus making it competitive with oil.

In addition, further cost benefits can be obtained by growing the algae and fungus on waste material, such as worm excrement castings from vermicomposting. In this way, expensive growth mediums will not have to be used. This will have the ancillary benefit of the algae and fungus cleaning up wastewater by absorbing toxic ions or pollutants from the water.

The fungal extraction of lipids from algae is the subject of a prior patent application by this inventor. The fungal assisted growth method in this application can also be combined with fungal extraction of the lipids in the algae in one continuous process. When the algae is “stressed” in the medium by depriving it of nutrients, it will no longer be able to provide the food source for the fungus. This will boost the production of oil in the algae cells due to the stressing; and will cause the fungus to attack the algae in order to survive. The fungus will then release the enzymes to break down the algae cell wall, and then the lipids from the algae can easily be obtained. This will efficiently lower the cost, energy requirements, and resources needed to sustainably make algae a viable fuel for the future. The method capitalizes on the symbiotic relationship of algae and fungus in each of the steps in the algae biofuel production process.

### BRIEF SUMMARY OF THE INVENTION

The new method uses adds fungus to the growth medium for algae in order to substantially increase the growth of algae.

The new method also uses waste material as an additional supplement to the growth medium

### DETAILED DESCRIPTION OF THE INVENTION

1. Obtain a small strain of the fungus, Aspergillus niger (“A. Niger”). Incubate the A. Niger at 30 degrees Celsius in a solution of potato dextrose agar, distilled water, ferrous sulfate, and magnesium sulfate. This will be the stock culture of the fungus.

2. Create a medium of 100 mL of BG11, 150 mL Distilled water, and 50 mL potato dextrose medium. Pour in a 16 oz plastic water bottle with the label removed. Repeat one more time for two bottles.

a. The potato dextrose medium (per 500 mL) contains: 20 g potato dextrose, 500 mL Distilled water, 2 g ferrous sulfate, and 2 g magnesium sulfate.

3. Pipette 1 mL of diluted fungal spores into the medium in each bottle. Poor 25 mL of concentrated algae cells into each solution.

4. Aerate one of the cultures. Screw the cap on the other culture. Place both cultures under a grow light with a 14:10 light to dark cycle. Keep the temperature at 20-24 degrees Celsius.

### Worm Casting Medium:

1. Place 200 g of Red (composting) Worm castings in 1 L of distilled water. Aerate for 24 hours. Strain the worm castings out using a cheese cloth.

2. Combine 100 mL of worm casting medium with 50 mL potato medium and 150 mL distilled water (described above) and repeat the same procedures as above.

### Coir Waste/Worm Casting Medium:

1. Place 100 g of coir fiber/pith mixture and 100 g worm castings in 1 L of distilled water. Aerate for 24 hours. Remove solids with a cheesecloth.

2. Combine 150 mL of medium with 150 mL distilled water and repeat the same procedures as above.

It turns out the cultures can grow with aeration and without. The overall idea is growing the fungus and algae together. The algae and fungus could grow in all three growth mediums described above. (BG11/Potato; Worm Casting/Potato; Coir Waste/Worm Casting). The two organisms can essentially grow on anything together, as long as glucose/cellulose, nitrates, and phosphorous are provided.

1. A method that uses fungus to assist in the growth of algae.

2. A method that incorporates waste material as part of the growth medium for algae and fungus.

3. A method that can be combined with fungal extraction method of extracting lipids from algae in one continuous process.
